(19) United States
(12) Patent Application Publication
(10) Pub. No.: US 2005/0054897 A1
(43) Pub. Date: Mar. 10, 2005

(54) Capsule endoscope and capsule endoscope system

(75) Inventors: Masayuki Hashimoto, Tokyo (JP); Kazutaka Nakatsuchi, Tokyo (JP); Noriyuki Fujimori, Tokyo (JP)

Correspondence Address: SCULLY SCOTT MURPHY & PRESSER, PC, 400 GARDEN CITY PLAZA, GARDEN CITY, NY, 11530, US

(73) Assignee: OLYMPUS CORPORATION, TOKYO (JP)

(21) Appl. No.: 10/936,157

(22) Filed: Sep. 8, 2004

(30) Foreign Application Priority Data
Sep. 8, 2003 (JP) 2003-315542

(51) Int. Cl. A61B 1/04

(52) U.S. Cl. 600/118; 600/160

(57) ABSTRACT

A capsule endoscope includes a function executing unit which executes predetermined functions including capturing an image in a subject where the capsule endoscope is introduced; a package which encloses the capsule endoscope, and has an opening; and a cover which covers the opening, and is made of a material reacting with a substance in the subject to expose the opening. The capsule endoscope also includes a sensor which detects that the capsule endoscope is located in the subject, through the opening; and a driving controller which controls the function executing unit.

### CROSS-REFERENCE TO RELATED APPLICATIONS

This application claims the benefit of priority of Japanese Patent Application No. 2003-315542 filed on Sep. 8, 2003, and the disclosure of which is incorporated herein by its entirely.

### BACKGROUND OF THE INVENTION

1) Field of the Invention

The present invention relates to a capsule endoscope used in a state where the endoscope is introduced into a subject and executes predetermined functions in the subject, and a capsule endoscope system for processing image data transmitted from the capsule endoscope.

2) Description of the Related Art

Swallowing capsule endoscopes are known in the fields of endoscopes. The capsule endoscopes have an imaging function and a wireless communication function. While an capsule endoscope is swallowed through a mouth of a patient for observation (examination) and then comes out of a body naturally, the capsule endoscope moves in a body cavity including internal organs such as stomach and small intestine according to their peristaltic movement so as to successively capture their images.

Image data captured in a body by the capsule endoscope, while moving in the body cavity, are successively transmitted to an outside through wireless communication, and are stored in a memory of an external receiver. When a patient carries the receiver having the wireless communication function and the memory function, the patient can move freely even while the patient swallows the capsule endoscope and then the capsule comes out of the body. Thereafter, doctors or nurses make a display device to display images of organs based on the image data stored in the memory so as to be capable of making a diagnosis.

While a driving power of such capsule endoscopes may be fed from a built-in power supply, in recent years an attention is paid to a configuration in which the driving power is fed from the outside via wireless transmission to the capsule endoscopes. Such a configuration in which the power is fed from the outside can avoid whole power from being consumed involuntarily and the driving from being stopped when the capsule endoscope moves in a body cavity.

To control the driving of the capsule endoscope, the configuration, in which a reed switch for turning on/off the capsule endoscope according to an external magnetic field is provided into the capsule endoscope and a permanent magnet for applying a magnetic field is provided to a package that houses the capsule endoscope, is suggested. In other words, the reed switch provided into the capsule endoscope maintains an OFF state under an environment that the external magnetic field of certain strength or more is applied, and turns into an ON state when the strength of the external magnetic field is decreased. Therefore, the capsule endoscope is not driven in the state where it is housed in the package, whereas the capsule endoscope, when taken out of the package, is out of the influence of the permanent magnet and starts driving. Such a configuration can prevent the capsule endoscope from starting to be driven while they are housed in the package (for example, see Published International Application WO 01/35813).

Even when the mechanism that controls the driving state of the capsule endoscope is provided, however, the driving of the capsule endoscope that is outside a subject cannot be always prevented. In other words, since it takes certain time from when the capsule endoscope is taken out from the package to when the capsule endoscope is introduced into the subject, the capsule endoscope starts driving before it is introduced into the subject. A problem, which arises when the capsule endoscope starts driving before it is introduced into the subject, is explained below.

When the capsule endoscope starts driving before it is introduced into the subject, useless image data not used for diagnoses are obtained. The capsule endoscope is configured to start driving and capturing an image, and to wirelessly transmit the image data obtained. When the capsule endoscope is driven before it is introduced into the subject, operations including the capturing are performed on the outside of the subject.

As a result, a lot of image data are obtained while the capsule is unsealed and then the capsule endoscope is introduced into the subject, and thus the doctors or the like make diagnoses after the useless image data are deleted. Since an imaging rate of the general capsule endoscope is about two images per second, when the capsule endoscope is driven on the outside of the subject, a lot of unnecessary image data are obtained even for a short time of about a few dozen of seconds. To avoid obtaining such useless image data, therefore, it is necessary to prevent the capsule endoscope from starting driving before the capsule endoscope is introduced into the subject.

Since a certain amount of the driving power is required for obtaining such useless image data, when the capsule endoscope is driven on the outside of the subject, the electric power stored in the capsule endoscope is consumed away. Also from a viewpoint of the power consumption, therefore, it is necessary to prevent the capsule endoscope from starting driving before it is introduced into the subject.

It is necessary to check an operation of the capsule endoscope before it is taken orally, in which case it is desired to consume the bare minimum of electric power and suppress radiation of an unnecessary radio wave.

### SUMMARY OF THE INVENTION

A capsule endoscope according to one aspect of the present invention includes a function executing unit which executes predetermined functions including capturing an image in a subject where the capsule endoscope is introduced; a package which encloses the capsule endoscope, and has an opening; a cover which covers the opening, and is made of a material reacting with a substance in the subject to expose the opening; a sensor which detects that the capsule endoscope is located in the subject, through the opening; and a driving controller which controls the function executing unit.

A capsule endoscope system according to another aspect of the present invention includes a capsule endoscope introduced into a subject; and a receiving which is arranged out of the subject, and receives information obtained by the capsule endoscope, via wireless communication. The capsule endoscope includes a function executing unit which executes predetermined functions including capturing an image in a subject where the capsule endoscope is introduced; a package which encloses the capsule endoscope, and has an opening; a cover which covers the opening, and is made of a material reacting with a substance in the subject to expose the opening; a sensor which detects that the capsule endoscope is located in the subject, through the opening; a driving controller which controls the function executing unit; and a wireless transmitter which transmits information obtained by the function executing unit. The receiving device includes a wireless receiver which receives the information transmitted from the wireless transmitter; and a processor which analyzes the information received.

The other objects, features, and advantages of the present invention are specifically set forth in or will become apparent from the following detailed description of the invention when read in conjunction with the accompanying drawings.

### BRIEF DESCRIPTION OF THE DRAWINGS

FIG. 1 is a schematic diagram of a capsule endoscope system according to a first embodiment of the present invention;

FIG. 2 is a block diagram of a transmitting/receiving device of the capsule endoscope system shown in FIG. 1;

FIG. 3 is a block diagram of a capsule endoscope of the capsule endoscope system shown in FIG. 1;

FIG. 4 is a flowchart of a driving control procedure in the capsule endoscope by a driving controller;

FIG. 5 is a block diagram of a modification of the capsule endoscope according to the first embodiment;

FIG. 6 is a block diagram of another modification of the capsule endoscope according to the first embodiment;

FIG. 7 is a block diagram of a capsule endoscope according to a second embodiment of the present invention; and

FIG. 8 is a flowchart of a driving control procedure in the capsule endoscope by a driving controller shown in FIG. 7.

### DETAILED DESCRIPTION

Exemplary embodiments of a capsule endoscope and a capsule endoscope system relating to the present invention will be explained in detail below with reference to the accompanying drawings.

A capsule endoscope system according to a first embodiment is explained.

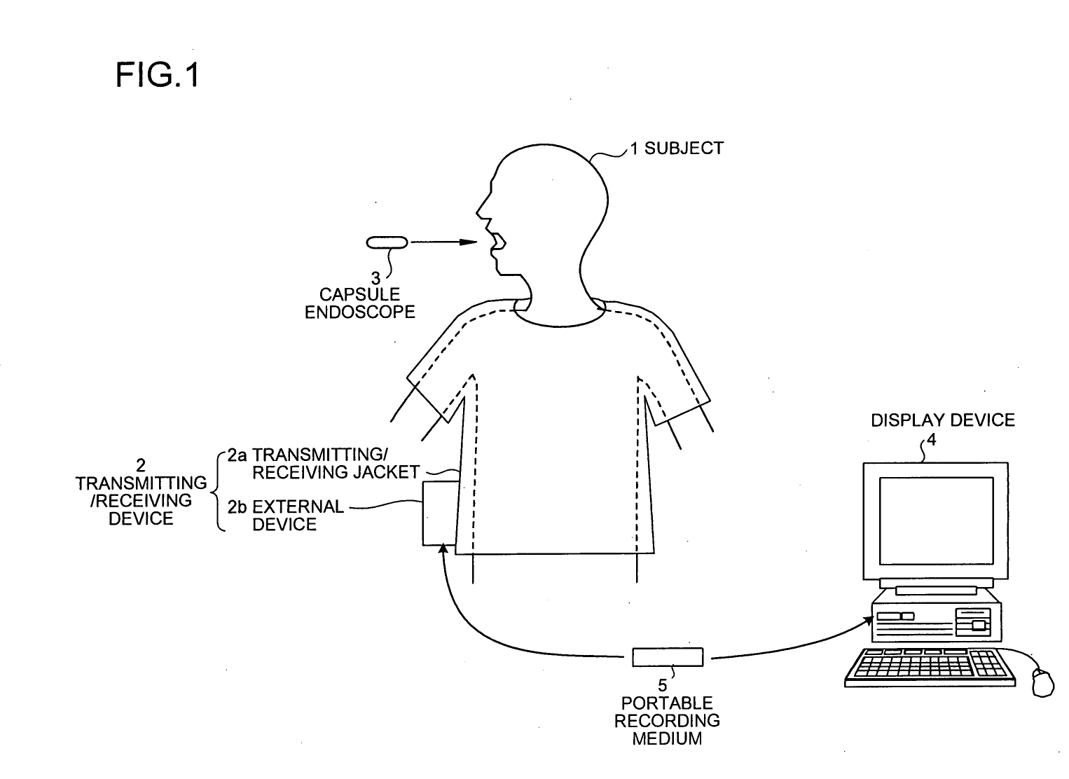

FIG. 1 is a schematic diagram of the capsule endoscope system according to the first embodiment. As shown in FIG. 1, the capsule endoscope system has a transmitting/receiving device 2 having a wireless transmitting/receiving function, and a capsule endoscope 3 that is introduced into a subject 1, is operated by driving power gotten by a radio signal transmitted from the transmitting/receiving device 2, and captures images in a body cavity so as to transmit image data to the transmitting/receiving device 2. Further, the capsule endoscope system has a display device 4 that displays images in the body cavity based on the data received by the transmitting/receiving device 2, and a portable recording medium 5 that transmits/receives the data between the transmitting/receiving device 2 and the display device 4. The transmitting/receiving device 2 has a transmitting/receiving jacket 2a worn by the subject 1, and an external device 2b that processes a radio signal transmitted/received via the transmitting/receiving jacket 2a.

The display device 4 is for displaying the images in the body cavity imaged by the capsule endoscope 3, and has a configuration of a work station or the like for displaying the images based on data gotten by the portable recording medium 5. Specifically, the display device 4 may have a configuration such that the images are displayed directly on a CRT display, a liquid crystal display or the like, or a configuration such that the images are output to another medium such as a printer.

The portable recording medium 5 is detachable from the external device 2b and the display device 4, and has a configuration such that when inserted to be attached to both of them, information can be output or recorded. Specifically, the portable recording medium 5 is inserted to be attached into the external device 2b so as to record data transmitted from the capsule endoscope 3 therein while the capsule endoscope 3 is moving in the body cavity of the subject 1. After the capsule endoscope 3 is ejected from the subject 1, namely, after the imaging of the inside of the subject 1 is completed, the portable recording medium 5 is taken out of the external device 2b and is inserted to be attached into the display device 4. The recorded data are read by the display device 4. The data are transmitted/received between the external device 2b and the display device 4 by the portable recording medium 5 such as a compact flash (registered trademark) memory. As a result, the subject 1 can move freely during the photographing of the body cavity unlike the case such that the external device 2b and the display device 4 are connected with each other by a wire.

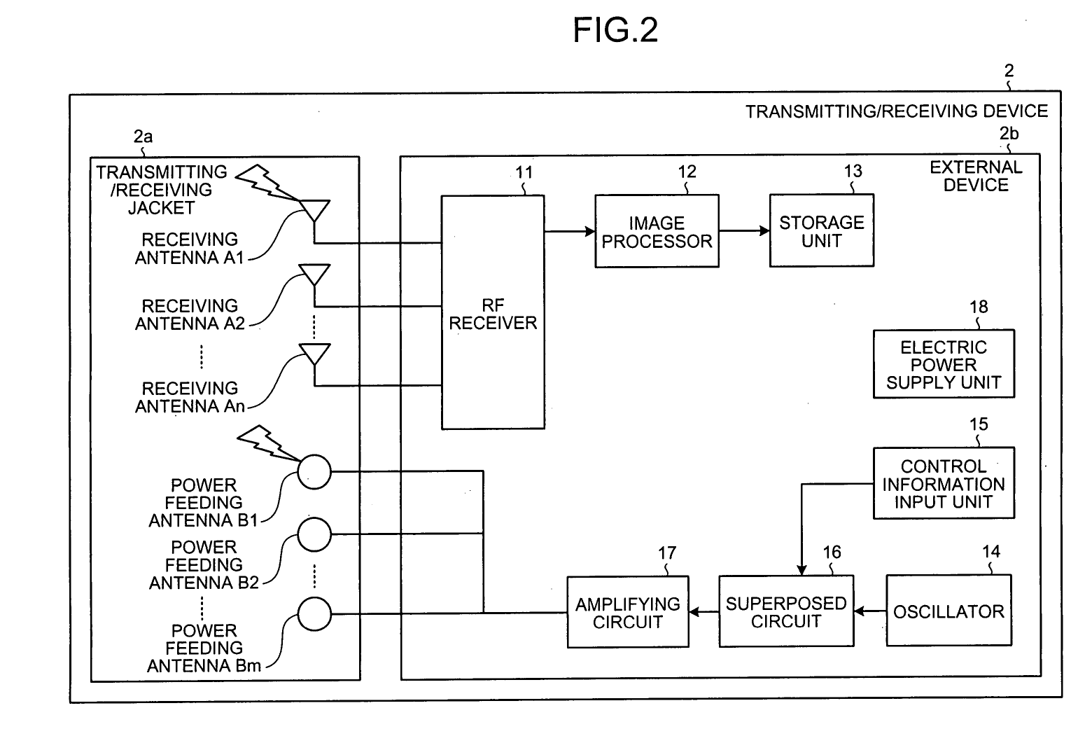

The transmitting/receiving device 2 has a function as a power feeding device for transmitting electric power to the capsule endoscope 3, and also a function as a receiving device that receives image data of the body cavity wirelessly transmitted from the capsule endoscope 3. FIG. 2 is a block diagram of the transmitting/receiving device 2. As shown in FIG. 2, the transmitting/receiving device 2 can be worn by the subject 1, and has the transmitting/receiving jacket 2a having receiving antennas A1 to An and power feeding antennas B1 to Bm, and the external device 2b that processes a transmitted/received radio signal.

The external device 2b has a function for processing the radio signal transmitted form the capsule endoscope 3. Specifically, as shown in FIG. 2, the external device 2b has an RF receiver 11 that executes a predetermined process such as decoding a radio signal received by the receiving antennas A1 to An and extracts image data gotten by the capsule endoscope 3 from the radio signal so as to output the image data, an image processor 12 that executes a process necessary for the output image data, and a storage unit 13 that records the image data executed the image process. The image data are recorded in the portable recording medium 5 via the storage unit 13.

The external device 2b has a function for generating a radio signal transmitted to the capsule endoscope 3. Specifically, the external device 2b has an oscillator 14 that generates a power feeding signal and defines an oscillation frequency, a control information input unit 15 that generates a control information signal for controlling a driving state of the capsule endoscope 3, a superposed circuit 16 that synthesizes the power feeding signal with the control information signal, and an amplifying circuit 17 that amplifies strength of the synthesized signal. The signal amplified by amplifying circuit 17 is transmitted to the power feeding antennas B1 to Bm so as to be transmitted to the capsule endoscope 3. The external device 2b includes a power supply unit 18 having a predetermined capacitor, an AC power source adapter or the like. The components of the external device 2b use electric power supplied from the power supply unit 18 as a driving energy.

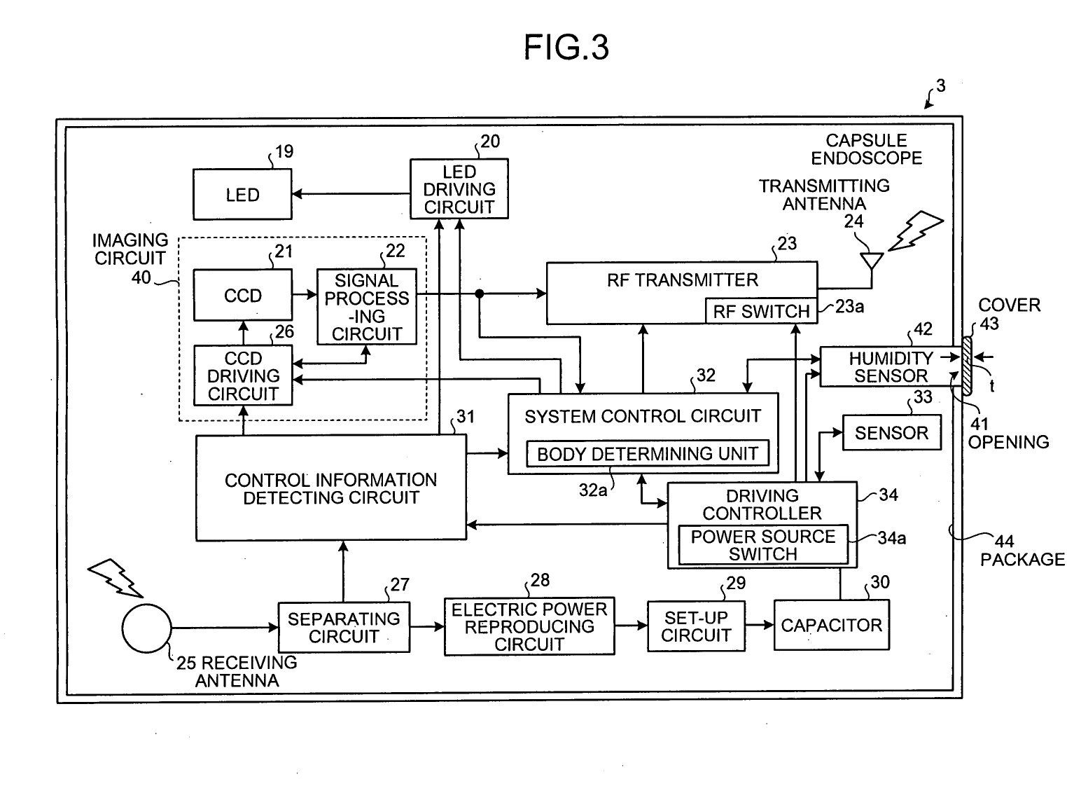

The capsule endoscope 3 is explained. FIG. 3 is a block diagram of a configuration of the capsule endoscope 3. As shown in FIG. 3, the capsule endoscope 3 has a light emitting diode (LED) 19 that irradiates an imaging area when the inside of the subject 1 is photographed, an LED driving circuit 20 that controls a driving state of the LED 19, a charge coupled device (CCD) 21 that images a reflected light image from the area irradiated by the LED 19, and a signal processing circuit 22 that processes an image signal output from the CCD 21 into imaging information having a desired format. Further, the capsule endoscope 3 has a CCD driving circuit 26 that controls a driving state of the CCD 21, an RF transmitter 23 that modulates the image data imaged by the CCD 21 by the signal processing circuit 22 so as to generate an RF signal, a transmitting antenna 24 that wirelessly transmits the RF signal output from the RF transmitter 23, and a system control circuit 32 that controls operations of the LED driving circuit 20, the CCD driving circuit 26, and the RF transmitter 23. The CCD 21, the signal processing circuit 22, and the CCD driving circuit 26 are collectively called as an imaging circuit 40.

When these mechanisms are provided while the capsule endoscope 3 is introduced into the subject 1, image information about portions to be examined irradiated by the LED 19 is acquired by the CCD 21. The signal processing circuit 22 executes the signal process on the acquired image information, and after the RF transmitter 23 converts the image information into an RF signal, it transmits the RF signal to the outside via the transmitting antenna 24.

The capsule endoscope 3 has a receiving antenna 25 that receives a radio signal transmitted from the transmitting/receiving device 2, and a separating circuit 27 that separates a power feeding signal from the signal received by the receiving antenna 25. Further, the capsule endoscope 3 has an electric power reproducing circuit 28 that reproduces electric power from the separated power feeding signal, a set-up circuit 29 that sets up the reproduced electric power, and a capacitor 30 that stores the set-up electric power. Further, the capsule endoscope 3 has a control information detecting circuit 31 that detects contents of a control information signal from a component separated from the power feeding signal by the separating circuit 27, and outputs a control signal to the LED driving circuit 20, the CCD driving circuit 22, and the system control circuit 32 if necessary. The control information detecting circuit 31 and the system control circuit 32 also have a function for distributing the driving power supplied form the capacitor 30 to another component.

The capsule endoscope 3 having these components receives a radio signal transmitted from the transmitting/receiving device 2 via the receiving antenna 25, and separates a power feeding signal and a control information signal from the received radio signal. The control information signal is output to the LED driving circuit 20, the CCD driving circuit 22, and the system control circuit 32 via the control information detection circuit 31 so as to be used for controlling the driving states of the LED 19, the CCD 21, and the RF transmitter 23. On the other hand, the electric power reproducing circuit 28 reproduces the power feeding signal as electric power, and the set-up circuit 29 sets up the reproduced electric power to a potential of the capacitor 30 so that the set-up electric power is stored in the capacitor 30. The capacitor 30 has a function for being capable of supplying electric power to the system control circuit 32 and the other components. The capsule endoscope 3 is configured so that electric power is supplied by wireless transmission from the transmitting/receiving device 2.

The capsule endoscope 3 has a sensor 33 that detects predetermined magnetic, light and wave signals and the like, a humidity sensor 42, and a driving controller 34 that control driving states of various function executing units such as the system control circuit 32, the RF transmitter 23, and the imaging circuit 40. The driving controller 34 has a power source switch 34a as a main switch for a power source of the entire capsule endoscope 3. The sensor 33 detects magnetism, light, wave and the like as signals for an on/off operation of the power source switch 34a, so as to output a detected result to the driving controller 34. The RF transmitter 23 has an RF switch 23a as a power source switch for the entire RF transmitter 23. The system control circuit 32 has a body determining unit 32a, and the body determining unit 32a determines whether humidity is humidity in a subject based on a detected result from the humidity sensor 42 so as to determine whether the capsule endoscope 3 is present in the subject or on the outside of the subject. When the driving controller 34 acquires a determined result from the body determining unit 32a is such that the capsule endoscope 3 is present in the subject, the RF switch 23a is turned into an ON state so as to actuate the RF transmitter 23.

The entire capsule endoscope 3 is covered with a package 44. The humidity sensor 42 is exposed from the capsule endoscope 3 via an opening 41 partially provided on the package 44. In a state, however, before the capsule endoscope 3 is used, a cover 43 is provided so as to externally cover the opening 41. The cover 43 is covered with a candy material. In the state before the capsule endoscope 3 is used, therefore, the humidity sensor 42 does not function. When the capsule endoscope 3 is introduced into the subject, the cover 43 as the candy material is gradually melted by salvia in the subject, and its thickness t becomes thinner. The cover 43 melts completely with time so that the opening 41 is exposed, and the humidity sensor 42 detects outer humidity. When the humidity sensor 42 detects the humidity in the subject, such as humidity (moisture) of the salvia and humidity (moisture) of gastric juice, the RF switch 23a is turned into the ON state so as to actuate the RF transmitter 23.

Since the opening 41 is not exposed to the outside as long as the capsule endoscope 3 is not introduced into a subject, the RF switch is not turned into the ON state on the outside of the subject, and thus electric power consumption is not consumed uselessly. Further, when the humidity determined by the body determining unit 32a is set to the humidity of salvia or the humidity of gastric juice, the RF switch can be operated into an ON state from a desired position in the subject. As a result, captured images in the desired position can be gotten with less consumption of power source capacity.

Instead of the humidity sensor 42, a pH sensor may be used. In this case, an ON time of the RF switch can be set according to a difference in pH of salvia and pH of gastric juice. The cover 43 is formed by the candy material, but it is not limited to this, and red food die or wafer may be used. Further, the cover 43 may be transparent. Further, the thickness of the cover 43 is set so that a time period for which the opening is exposed can be adjusted.

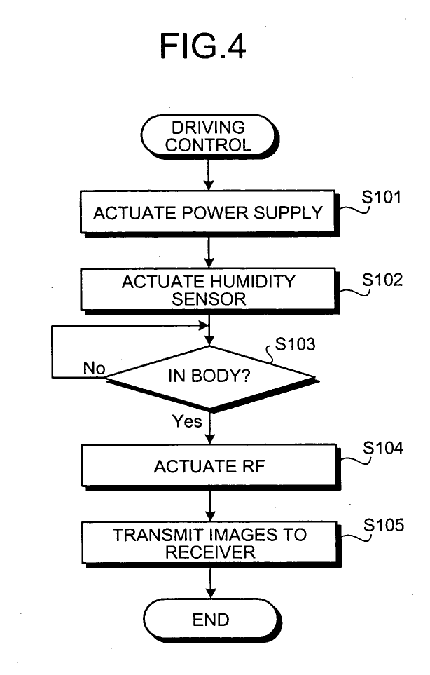

The driving control procedures of the respective units based on the determined result from the body determining unit 32a is explained with reference to FIG. 4. At the start time of the process, the components in the capsule endoscope 3 are in the OFF state. The sensor 33 does not require a power source, and detects a mechanical movement so as to brings the power source switch 34a into the ON/OFF state.

When the power source switch 34a is actuated (step S101), the driving controller 34 actuates at least the humidity sensor 43 (step S102). In this case, since the RF transmitter 23 is not actuated, even if the imaging circuit 40 is actuated, images gotten by the imaging circuit 40 are not transmitted to the outside.

Thereafter, the driving controller 34 determines whether a determined result such that the humidity sensor 42 is in a subject is gotten from the body determining unit 32a (step S103). When, for example, the humidity detected by the humidity sensor 42 is set as the humidity of salvia, it is determined that the humidity sensor 42 is in the subject when the humidity of salvia is detected. When not determined that the body determining unit 32a is in the subject (No at step S103), the driving controller 34 repeats the determining process at step S103.

On the other hand, when the body determining unit 32a determines that the capsule endoscope 3 is in the subject (Yes at step S103), it brings the RF switch 23a of the RF transmitter 23 into the ON state so as to actuate the RF transmitter 23 (step S104). When the imaging circuit 40 is not actuated then, it is actuated. After the RF transmitter 23 is actuated (step S104), image data imaged by the imaging circuit 40 are transmitted to the external device 2b via the RF transmitter 23 and the transmitting antenna 24 (step S105) so that the process is ended. A reception mechanism provided to the transmitting/receiving jacket 2a receives the transmitted image data, and supplies them to the display device 4 via the portable recording medium 5. The image data are displayed as subject images on a screen of the display device 4.

In the first embodiment, when the body determining unit 32a determines that the capsule endoscope 3 is in the subject, it brings the RF switch 23 in the OFF state into the ON state, so that captured images are transmitted. When the power source switch 34a is brought into the ON state, however, it may actuate the RF transmitter 23 in a low-power consumption mode, and when the body determining unit 32a determines that the capsule endoscope 3 is in the subject, it may change the RF transmitter 23 into a normal power consumption mode.

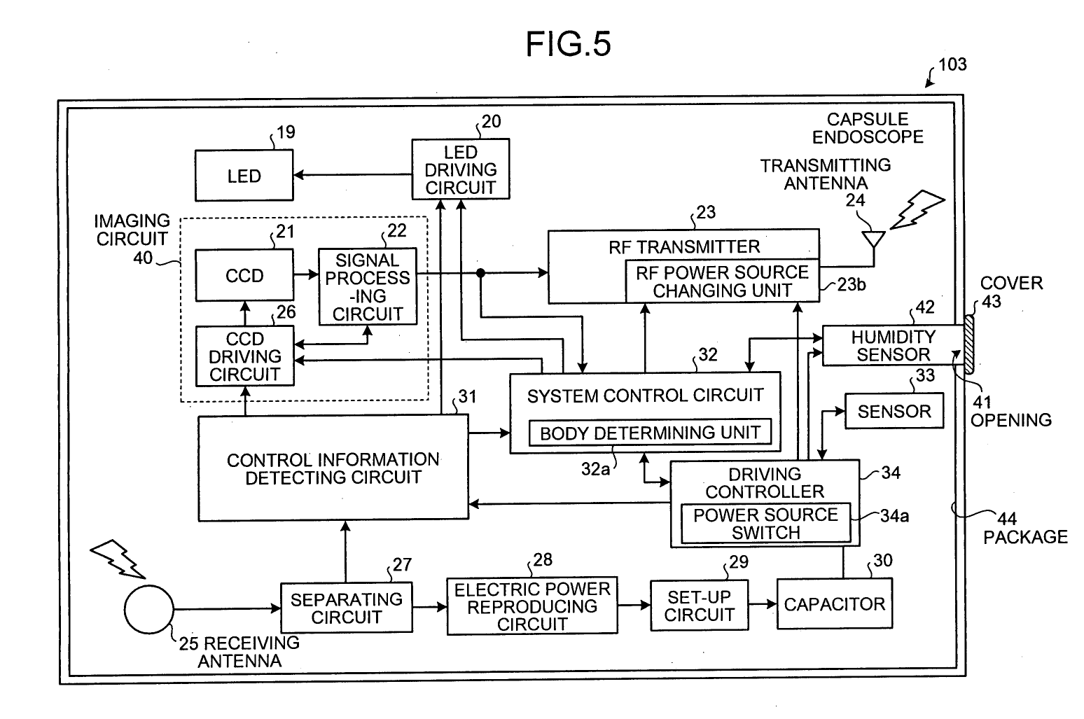

In this case, as shown in a capsule endoscope 103 in FIG. 5, the RF transmitter 23 has an RF power source changing unit 23b instead of the RF switch 23a. When the body determining unit 32a determines that the capsule endoscope 103 is out of the subject, the RF power source changing unit 23b sets the RF transmitter 23 into the low power consumption mode where it is driven by a lower power consumption than that in the normal state according to an instruction from the driving controller 34. When the body determining unit 32a determines that the capsule endoscope 103 is in the subject, the RF power source changing unit 23b changes the RF transmitter 23 into the normal power consumption mode where it is driven by the normal power consumption. As a result, a weak radio wave is transmitted from the transmitting antenna 24 in the low power consumption mode, and a radio wave having strength that can be received on the outside of the subject is output from the transmitting antenna 24 in the normal power consumption mode. When the capsule endoscope 103 is out of the subject, its operation is occasionally checked, and in this case, imaged data can be gotten by transmitting the weak radio wave. Therefore, the operation of the imaging circuit 40 can be checked.

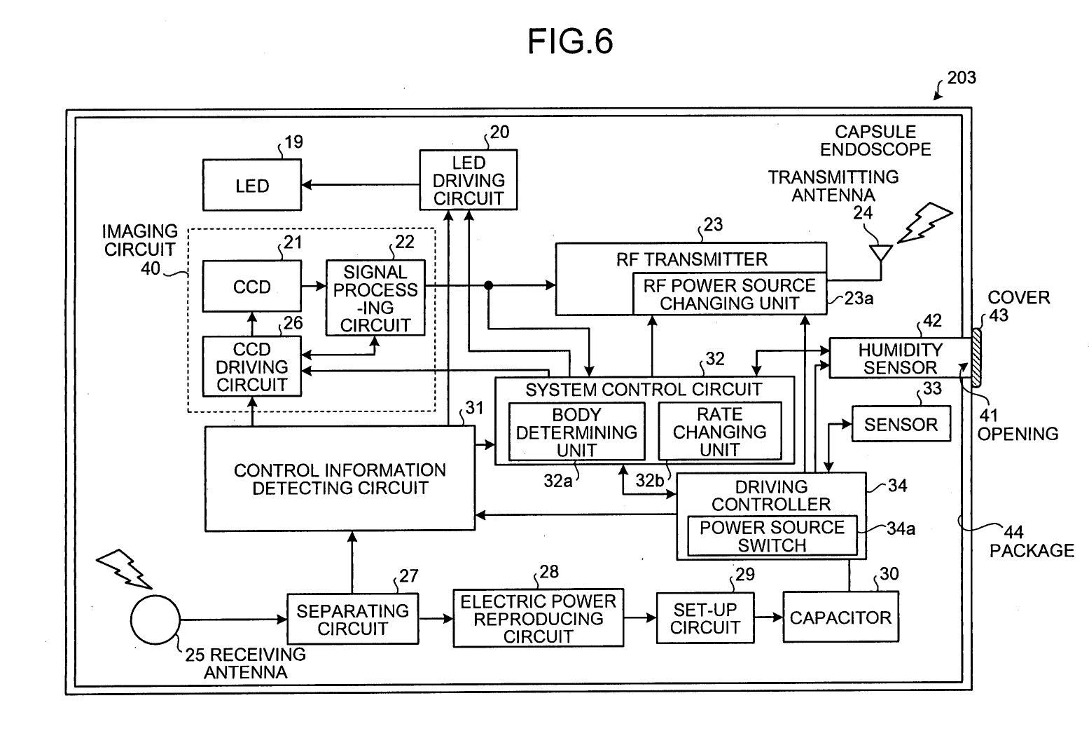

As shown in a capsule endoscope 203 in FIG. 6, a rate changing unit 32b may be provided, and when the body determining unit 32a determines that the capsule endoscope 203 is in the subject, the rate changing unit 32b decreases an imaging frame rate of the imaging circuit 40. When the body determining unit 32a determines that the capsule endoscope 203 is out of the subject, the rate changing unit 32b changes the imaging frame rate of the imaging circuit 40 into a normal rate. In FIG. 6, the RF power source changing unit 23b is used to further suppress radio wave radiation, thereby suppressing the power consumption. The power consumption can be, however, suppressed only by reducing the imaging frame rate.

In the first embodiment, with the provision of the cover 43, when the capsule endoscope 3 (103, or 203) is introduced into the subject securely, the humidity sensor 42 is operated, so that useless power consumption is prevented when the capsule endoscope 3 (103, or 203) is placed out of the subject. Further, the provision of the humidity sensor 42 enables desired images of desired positions in the subject imaged and transmitted.

A second embodiment of the present invention is explained below. In the first embodiment, the function of the humidity sensor 42 brings the components such as the RF switch 23a that consume a lot of power consumption into the operating state. In the second embodiment, however, the RF switch and the like can also be brought into the ON state in a desired position finely determined.

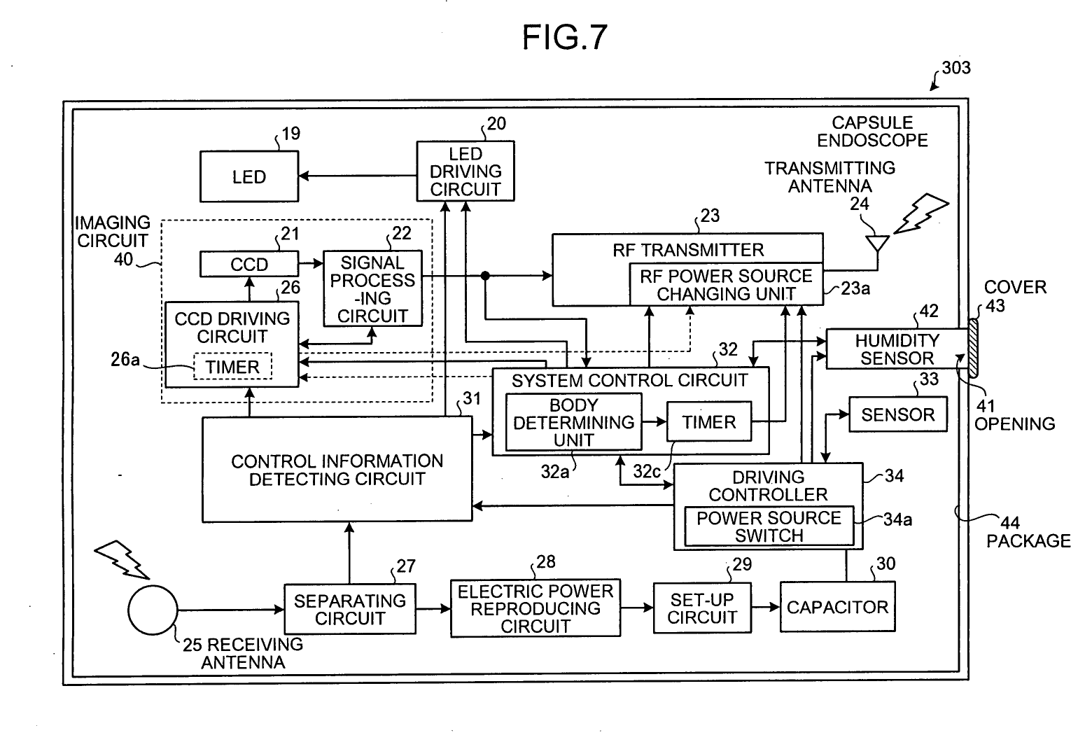

FIG. 7 is a block diagram of a configuration of the capsule endoscope according to the second embodiment. In the capsule endoscope 303 shown in FIG. 7, in addition to the configuration of the capsule endoscope shown in FIG. 3, a timer 32c is provided. When the body determining unit 32a determines that the capsule endoscope 303 is in a subject, the timer 32c starts to count predetermined time so as to actuate the RF switch 23a when the predetermined time comes. In this case, the body determining unit 32a can determine that the position of the capsule endoscope 303 is in the subject, but the position is basically limited to an area such as a stomach where characteristic images can be gotten. It is occasionally desired that the data of the gotten Images is transmitted from a middle position of the area. In this occasion, the RF transmitter 23 can be brought into the normal transmission state from a finely desired position, and the data of the gotten images can be transmitted to the external device 2b. The timer 32c is provided to the system control circuit 32, but the position is not limited to this, it can be provided into the CCD driving circuit 26 having a timing generator or the like so that the configuration of the CCD driving circuit 26 is used effectively.

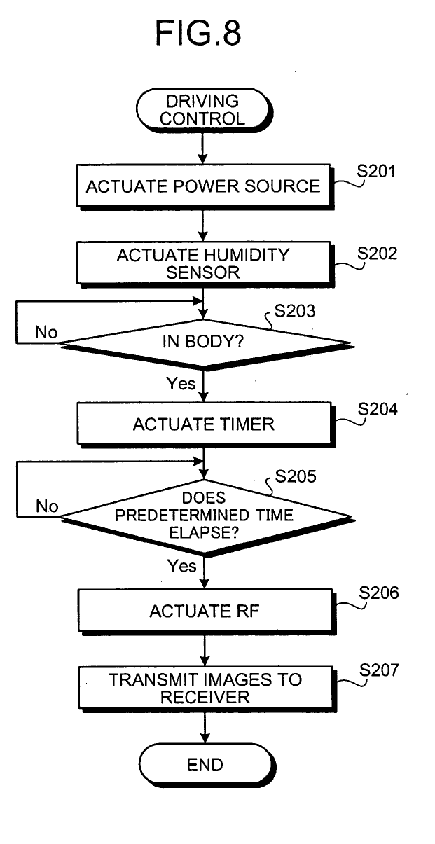

The driving control procedure of the respective units based on the determined result of the body determining unit 32a is explained with reference to a flowchart shown in FIG. 8. At the start time of this process, the components of the capsule endoscope 303 are in the OFF state. The sensor 33 does not require a power source, and detects, for example, a mechanical movement so as to bring the power source switch 34a into the ON/OFF state.

When the power source switch 34a is actuated (step S201), the driving controller 34 actuates at least the humidity sensor 42 (step S202). Thereafter, the driving controller 34 determines whether the determined result that the humidity sensor 42 is in a subject is gotten from the body determining unit 32a (step S203). When, for example, the humidity determined by the humidity sensor 42 is set to the humidity of salvia, it is determined that the humidity sensor 42 is in the subject when the humidity of salvia is detected. When the body determining unit 32a does not determine that the humidity sensor 42 is in the subject (No at step S203), the driving controller 34 repeats the determining process at step S203.

On the other hand, when the body determining unit 32a determines that the capsule endoscope 303 is in the subject (Yes at step S203), it actuates the timer 32c (step S204). Thereafter, the time counted by the timer 32c runs beyond the predetermined time (step S205). When the predetermined time does not elapse (No at step S205), the determining process at step S205 is repeated. On the other hand, when the predetermined time elapses (YES at step S205), the RF switch 23a of the RF transmitter 23 is brought into the ON state, so as to actuate the RF transmitter 23 (step S206). When the imaging circuit 40 is not actuated then, it is actuated. After the RF transmitter 23 is actuated, the image data imaged by the imaging circuit 40 are transmitted to the external device 2b via the RF transmitter 23 and the transmitting antenna 24 (step S207), and the process is ended. The receiving mechanism provided to the transmitting/receiving jacket 2a receives the transmitted image data, and supplies them to the display device 4 via the portable recording medium 5 so that the image data are displayed as the images of the inside of the subject on the screen of the display device 4.

In the second embodiment, in addition to the effects of the first embodiment, the RF switch 23a or the like can be actuated from a position of the subject finely determined, so that minimum necessary images can be gotten and the power consumption can be reduced to a minimum necessary level.

In the first and the second embodiments, the cover 43 is formed by, for example, a candy material that reacts to a substance in a path of the subject, through which the capsule endoscope 3 (103, 203, or 303) passes and which contacts with the capsule endoscope 3 (103, 203, or 303). The coating material is not limited to this, and a coating material that melts in water can be used when the substance in the subject is water used via the oral route. Further, a coating material that reacts to a predisposing material that is introduced into a stomach in advance can be used. In this case, the cover 43 melts in the stomach so that the opening 41 is exposed.

Although the invention has been described with respect to a specific embodiment for a complete and clear disclosure, the appended claims are not to be thus limited but are to be construed as embodying all modifications and alternative constructions that may occur to one skilled in the art which fairly fall within the basic teaching herein set forth.

1. A capsule endoscope comprising:
a function executing unit which executes predetermined functions including capturing an image in a subject where the capsule endoscope is introduced;
a package which encloses the capsule endoscope, and has an opening;
a cover which covers the opening, and is made of a material reacting with a substance in the subject to expose the opening;
a sensor which detects that the capsule endoscope is located in the subject, through the opening; and
a driving controller which controls the function executing unit.

2. The capsule endoscope according to claim 1, wherein the cover melts in the substance in the subject.

3. The capsule endoscope according to claim 2, wherein the cover is made of a candy material.

4. The capsule endoscope according to claim 2, wherein the cover is made of a red food die.

5. The capsule endoscope according to claim 2, wherein the cover is made of a wafer.

6. The capsule endoscope according to claim 1, wherein the cover exposes the opening after the cover reacts with the substance in the subject for a predetermined time period.

7. The capsule endoscope according to claim 6, wherein the predetermined time depends on a thickness of the cover.

8. The capsule endoscope according to claim 1, wherein the sensor detects predetermined characteristics of the substance in the subject.

9. The capsule endoscope according to claim 1, wherein the sensor is a humidity sensor.

10. The capsule endoscope according to claim 1, wherein the sensor is a pH sensor.

11. The capsule endoscope according to claim 1, further comprising a frame rate changing unit which changes between a first imaging process and a second imaging process, the first imaging process including capturing an image at a first frame rate, the second imaging process including capturing an image at a second frame rate higher than the first frame rate, wherein
the driving controller causes the frame rate changing unit to change the first imaging process to the second imaging process, when the sensor detects that the capsule endoscope is located in the subject.

12. The capsule endoscope according to claim 1, wherein
the function executing unit includes a wireless transmitter which transmits image data obtained by the capturing and which has a wireless actuating switch for the actuation of the wireless transmitter,
the driving controller switches the wireless actuating switch on, when the sensor detects that the capsule endoscope is located in the subject.

13. The capsule endoscope according to claim 12, further comprising a wireless power source changing unit which switches between a low power consumption mode and a normal power consumption mode, wherein
the driving controller causes the wireless power source changing unit to switch from the low power consumption mode to the normal power consumption mode, when the sensor detects that the capsule endoscope is located in the subject.

14. The capsule endoscope according to claim 12, further comprising a timer which counts predetermined time after the sensor detects that the capsule endoscope is located in the subject, and switches the wireless actuating switch on when the timer counts up the predetermined time.

15. The capsule endoscope according to claim 14, wherein the timer is provided into an imaging circuit.

16. The capsule endoscope according to claim 14, further comprising a wireless power source changing unit which switches between a low power consumption mode and a normal power consumption mode, wherein
the driving controller causes the wireless power source changing unit to switch from the low power consumption mode to the normal power consumption mode, when the timer counts up the predetermined time.

17. The capsule endoscope according to claim 14, further comprising a frame rate changing unit which changes between a first imaging process and a second imaging process, the first imaging process including capturing an image at a first frame rate, the second imaging process including capturing an image at a second frame rate higher than the first frame rate, wherein
the driving controller causes the frame rate changing unit to change the first imaging process to the second imaging process, when the timer counts up the predetermined time.

18. A capsule endoscope system comprising:
a capsule endoscope introduced into a subject; and
a receiving which is arranged out of the subject, and receives information obtained by the capsule endoscope, via wireless communication, wherein
the capsule endoscope includes
a function executing unit which executes predetermined functions including capturing an image in a subject where the capsule endoscope is introduced;
a package which encloses the capsule endoscope, and has an opening;
a cover which covers the opening, and is made of a material reacting with a substance in the subject to expose the opening;
a sensor which detects that the capsule endoscope is located in the subject, through the opening;
a driving controller which controls the function executing unit; and
a wireless transmitter which transmits information obtained by the function executing unit,
the receiving device includes
a wireless receiver which receives the information transmitted from the wireless transmitter; and
a processor which analyzes the information received.

19. The capsule endoscope system according to claim 18, wherein the driving controller switches a switch for the actuation of the wireless transmitter on, when the sensor detects that the capsule endoscope is located in the subject.
